(19) United States
(12) Patent Application Publication
(10) Pub. No.: US 2011/0054578 A1
(43) Pub. Date: Mar. 3, 2011

(54) HIGH-FREQUENCY ELECTRODE UNIT FOR SCALP CARE

(75) Inventors: Dae Young Pyo, Incheon (KR); Han Cheol Yoo, Incheon (KR)

(73) Assignees: Dae Young PYO, Incheon (KR); RevecoMED Research and Development, Inc., Fullerton, CA (US)

(21) Appl. No.: 12/552,716

(22) Filed: Sep. 2, 2009

(51) Int. Cl. A61N 1/04

(52) U.S. Cl. 607/115

(57) ABSTRACT

The present invention relates to an electrode unit for scalp care and a high-frequency treatment apparatus for scalp care including an electrode unit for scalp care. The present invention includes an electrode unit for scalp care which maximally applies a high-frequency current to a scalp of a user to be treated through a plurality of protruding terminals and efficiently stimulates the scalp through the plurality of protruding terminals. The inventive electrode unit for scalp care supplies the high-frequency current to the scalp to be treated through a plurality of protruding terminals so that the protruding terminals and the scalp can be easily come into direct close contact with each other, and stimulates the scalp, thereby maximizing a hair and scalp care effect.

### BACKGROUND OF THE INVENTION

(a) Field of the Invention

The present invention relates to a high-frequency treatment apparatus, and more particularly, to an electrode unit for scalp care which is adapted to apply a high-frequency current to a scalp to generate deep heat within the scalp and promote hair growth using the generated deep heat.

(b) Background Art

In general, a high-frequency treatment apparatus is a medical equipment which allows an high frequency current to be applied to the human body to supply electrical energy to it so that the supplied electrical energy is converted into biological energy, and promotes lipid metabolism and myokinesis through the deep heat generated by such biological energy so as to be used in obesity treatment, muscle strengthening, skin care, hair growth promotion, pain relief or the like.

When the high-frequency current is applied to the human body's tissues, its pulse width is very narrow, and thus ion movement hardly occur as well as electrochemical reaction or electrolysis does not occur. When such a high-frequency current is conducted into the human body, its tissues produce heat which is called “deep heat”. The reason for this is that whenever the direction of the current varies with time upon application of the electrical energy of a high frequency to the human body, the vibration of molecules constituting the human body's tissues induces the friction between the molecules to cause rotation, twisting and collision thereof to thereby produce biological heat.

The high-frequency current can heat only a specific site within the body tissues even without causing an unpleasant feeling or a muscle shrink in the human body, and the resultantly produced deep heat serves to raise the temperature of the body tissues so as to enhance the function of cells, increase the blood flow rate, and the like. As a consequence, the metabolism and the myokinesis of the human body are promoted so that the desired effects, for instance, such as obesity treatment, muscle strengthening, skin care, hair growth promotion, pain relief or the like, can be achieved.

FIG. 1 is a top perspective view illustrating a conventional high-frequency treatment apparatus according to the prior art.

Referring to FIG. 1, the conventional high-frequency treatment apparatus includes a main body 10 for generating a high-frequency current, a treatment electrode section 20 electrically connected to the main body, the treatment electrode section being configured to contact or touch an affected part of a user to be treated so as to apply the high-frequency current generated from the main body 10 to the affected part, a counter electrode section 30 electrically connected to the main body, the counter electrode section being configured to contact another body part of the user so as to allow the high-frequency current to be conducted to the contacted other body part of the user. In this case, the treatment electrode section 20 includes a treatment electrode unit 21, a handle 23 and a first cable 25, and the counter electrode section 30 includes a counter electrode plate 31 and a second cable 33.

After the treatment electrode unit 21 of the treatment electrode unit 30 comes into close contact with an affected part of a user to be treated, and the counter electrode plate 31 of the counter electrode section 30 comes into close contact with another body part of the user, the main body 10 is operated. Then, upon application of power to the main body 10, a high-frequency current is generated in the main body 10, and is conducted into the skin tissues of the human body of the user in close contact with the treatment electrode section 20 and the counter electrode section 30 so that the somatological heat or the deep heat is produced around the affected part in close contact with the treatment electrode unit 21.

The treatment electrode unit 21 of the high-frequency treatment apparatus as mentioned above is formed in a flat plate shape and is manufactured to be applied to all the body parts having a flat skin contact surface without being limited to a specific body part.

Thus, in case where it is desired to perform scalp care using the conventional high-frequency treatment apparatus including the treatment electrode unit 21, although a user tries to allow the flat plate-shaped treatment electrode unit 21 to come into close contact with a scalp to be treated, it is difficult for the treatment electrode unit 21 to directly contact the scalp due to hair protrudingly grown on the scalp. In addition, even if the treatment electrode unit 21 comes into close contact with the scalp, the skin contact area is small, which results in a reduction in the area where the deep heat is produced. Therefore, there occurs a problem in that the scalp stimulation is not performed efficiently and much time is spent treating the entire scalp.

The information disclosed in this background of the invention section is only for enhancement of understanding of the background of the invention and should not be taken as an acknowledgement or any form of suggestion that this information forms the prior art that is already known to a person skilled in the art.

### SUMMARY OF THE INVENTION

Accordingly, the present invention has been made in an effort to solve the aforementioned problems occurring in the prior art, and it is an object of the present invention to provide an electrode unit for scalp care which maximizes the contact area with the scalp so as to allow a high-frequency current to be smoothly transferred to the scalp to efficiently stimulate the scalp, thereby reducing the treatment time.

Another object of the present invention is to provide a high-frequency treatment apparatus for scalp care including an electrode unit for scalp care which maximizes the contact area with the scalp so as to allow a high-frequency current to be smoothly transferred to the scalp.

To accomplish the above object, in one aspect, the present invention provides an electrode unit for scalp care which is adapted to comes into close contact with a scalp of a user to be treated and apply a high-frequency current to the scalp so as to produce deep heat and treat the scalp by using the produced deep heat, the electrode unit including: a flat-shaped base electrode plate; a connecting terminal which is formed at one side of the base electrode plate and to which the high-frequency current is inputted; a plurality of protruding terminals which is formed at the other side of the base electrode plate and is adapted to allow the high-frequency current inputted to the connecting terminal to be applied to the scalp therethrough; and a first insulator adapted to surround the base electrode plate. Each of the plurality of protruding terminals includes a conductor and a second insulator adapted to surround the conductor.

To accomplish the above object, in another aspect, the present invention provides a high-frequency treatment apparatus for scalp care, including: a high-frequency generation unit for generating a high-frequency current: a first electrode unit electrically connected to the high-frequency generation unit, the first electrode unit including a plurality of protruding terminals adapted to come into close contact with a scalp of a user to be treated and being configured to apply the high-frequency current generated from the high-frequency generation unit to the scalp through the plurality of protruding terminals; and a second electrode unit electrically connected to the high-frequency generation unit, the second electrode unit including an electrode plate adapted to come into close contact with another body part of the user and being configured to allow the high-frequency current applied to the scalp to be conducted to the contacted other body part of the user through the electrode plate.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a top perspective view illustrating a conventional high-frequency treatment apparatus according to the prior art;

FIG. 2 is a top perspective view illustrating a high-frequency treatment apparatus for scalp care according to one preferred embodiment of the present invention;

FIG. 3 is a schematic functional block diagram illustrating the construction of a high-frequency treatment for scalp care apparatus according to one preferred embodiment of the present invention;

FIG. 4 is an exploded perspective view illustrating a first electrode unit of the present invention;

FIG. 5a is an exploded perspective view illustrating a head portion of the present invention, FIG. 5b is an assembled perspective view illustrating a head portion of the present invention, and FIG. 5c is an assembled perspective view illustrating a modification example of a head portion of the present invention;

FIG. 6a is a schematic vertical cross-sectional view illustrating a head portion of the present invention, and FIG. 6b is a schematic vertical cross-sectional view illustrating a plurality of protruding terminals of the head portion of the present invention;

FIG. 7a is a top plan view illustrating an upper case of a first insulator of the head portion of the present invention, FIG. 7b is a side view illustrating the upper case of the first insulator of the present invention, FIG. 7c is a top plan view illustrating a lower upper case of the first insulator of the present invention, and FIG. 7d is a top plan view illustrating a base electrode plate of the head portion of the present invention; and

FIG. 8 is a top perspective view illustrating a high-frequency treatment apparatus for scalp care according to another preferred embodiment of the present invention.

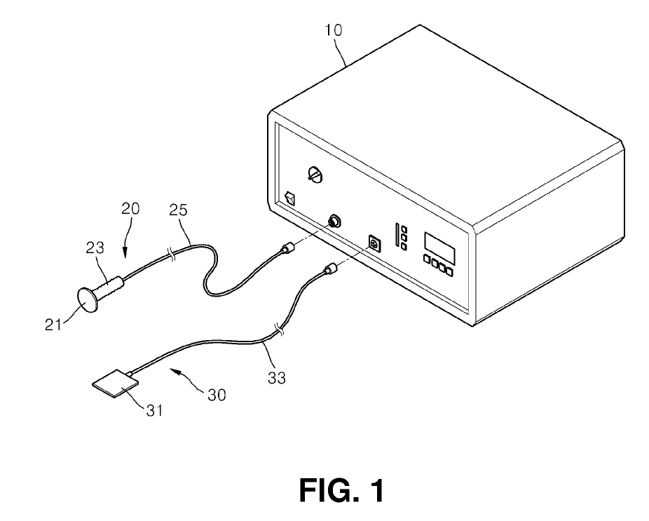

### DETAILED DESCRIPTION

The preferred embodiments of the present invention will now be made in detail with reference to the accompanying drawings, examples of which are illustrated in the drawings attached hereinafter, wherein like reference numerals refer to like elements throughout. In the detailed description of the preferred embodiments of the present invention, the size, the shape or the like of constituent elements may be exaggeratedly shown in the drawings for the sake of clarity and convenience of explanation.

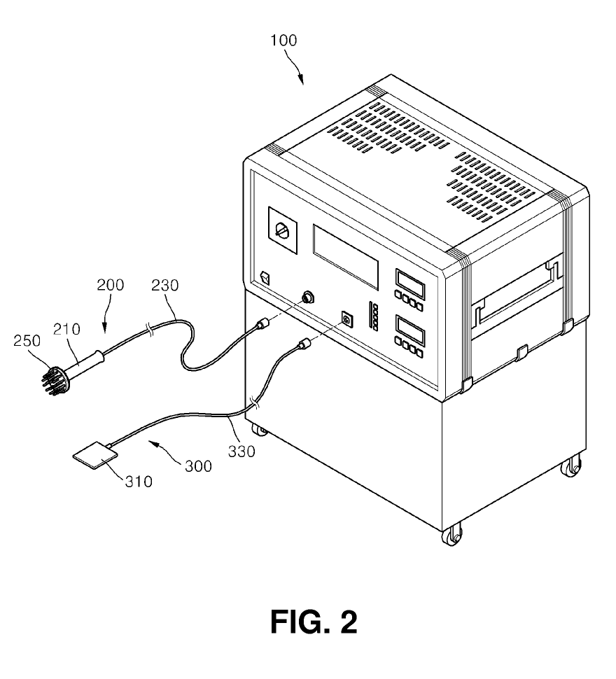

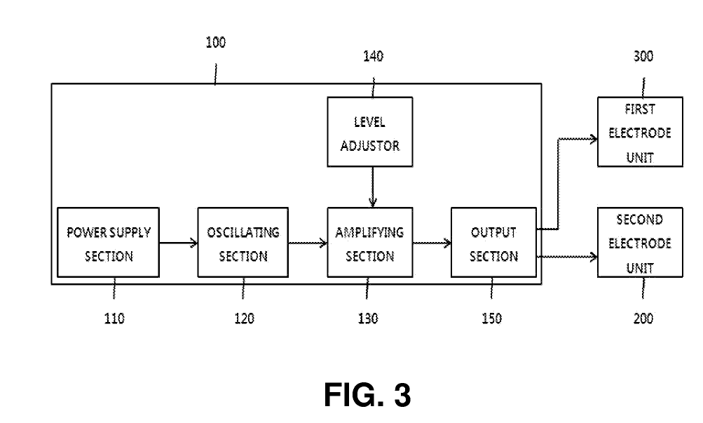

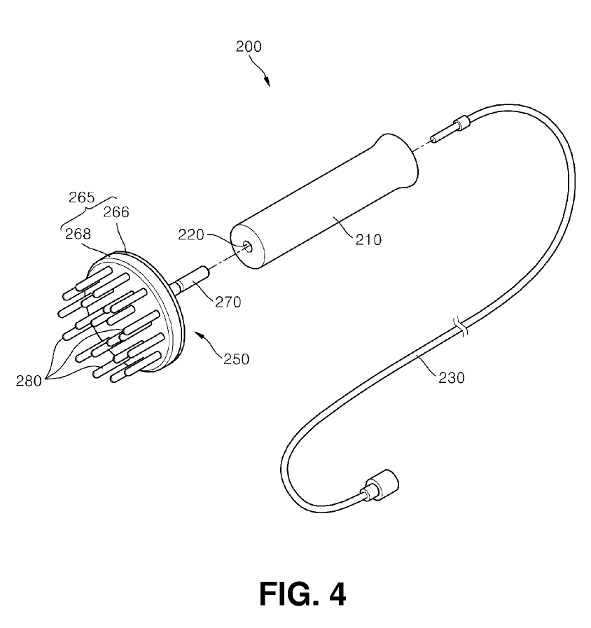

FIG. 2 is a top perspective view illustrating a high-frequency treatment apparatus for scalp care according to one preferred embodiment of the present invention, FIG. 3 is a schematic functional block diagram illustrating the construction of a high-frequency treatment for scalp care apparatus according to one preferred embodiment of the present invention, and FIG. 4 is an exploded perspective view illustrating a first electrode unit of the present invention.

Referring to FIGS. 2 to 4, the high-frequency treatment apparatus for scalp care according to one preferred embodiment of the present invention includes: a high-frequency generation unit 100 for generating a high-frequency current: a first electrode unit 200 electrically connected to the high-frequency generation unit, the first electrode unit including a plurality of protruding terminals adapted to come into close contact with a scalp of a user to be treated and being configured to apply the high-frequency current generated from the high-frequency generation unit to the scalp through the plurality of protruding terminals; and a second electrode unit 300 electrically connected to the high-frequency generation unit, the second electrode unit including an electrode plate adapted to come into close contact with another body part of the user and being configured to allow the high-frequency current applied to the scalp to be conducted to the contacted other body part of the user through the electrode plate.

The high-frequency generation unit 100 includes a power supply section 110, an oscillating section 120, an amplifying section 130, a level adjustor 140 and an output section 150.

The power supply section 110 serves to supply necessary power to respective constituent elements of the high-frequency generation unit 100. The oscillating section 120 serves to oscillate a high-frequency current, and the amplifying section 130 functions to amplify the high-frequency current oscillated in the oscillating section 120. The level adjustor 140 functions to adjust the intensity of the high-frequency current, and the output section 150 serves to output a high-frequency current of a predetermined intensity. The high-frequency current outputted from the output section 150 has a frequency of 400 kHz to 600 kHz. The high-frequency current of this frequency band is applied to the scalp of a subject to be treated through the first electrode unit 200 and the second electrode unit 300.

The first electrode unit 200 includes a body portion 210, a first cable 230 and a head portion 250, and the second electrode unit 300 includes a second electrode plate 310 and a second cable 330.

The body portion 210 of the first electrode unit 200 is formed in a stick shape for a convenient grip of a user. The body portion 210 is made of an insulating material, and has a through-hole formed at the inner center thereof. The first cable 230 is insertingly engaged with one end of the body portion 210 and the head portion 250 is insertingly engaged with the other end of the body portion 210.

The head portion 250 is a constituent element adapted to come into close contact with a scalp of a user to be treated so as to apply the high-frequency current generated from the high-frequency generation unit to the scalp. The head portion 250 includes a base electrode plate (not shown), a first insulator 265, a connecting terminal 270 and a plurality of protruding terminals 280.

One end of the connecting terminal 270 is engaged with one side of the base electrode plate and the plurality of protruding terminals 280 is engaged with other side of the base electrode plate. The first insulator 265 is constructed to surround the base electrode plate. The other end of the connecting terminal 270 is inserted into the through-hole formed at the inner center of the body portion so as to be electrically connected to the first cable 230.

The second electrode unit 300 includes a second electrode plate 310 adapted to come into close contact with another body part of the user, for example, arms, hands or the like, except the scalp and a second cable 330 for interconnecting the second electrode plate 310 and the output section 150 of the high-frequency generation unit 100. The second cable 330 is electrically connected at one end thereof to the output section 150 and is electrically connected at the other end thereof to the second electrode plate 310. The second electrode plate 310 is made of a conductive material. Although the present invention has described that the second electrode plate 310 is formed in a square plate shape, but is not limited thereto and may be formed in various shapes.

Preferably, the second electrode plate 310 is operated as a ground terminal. For example, in correspondence to the high-frequency current applied to the human body through the first electrode unit 200, when a positive current is applied to the human body through the first electrode unit 200, the second electrode plate 310 is operated as a negative terminal.

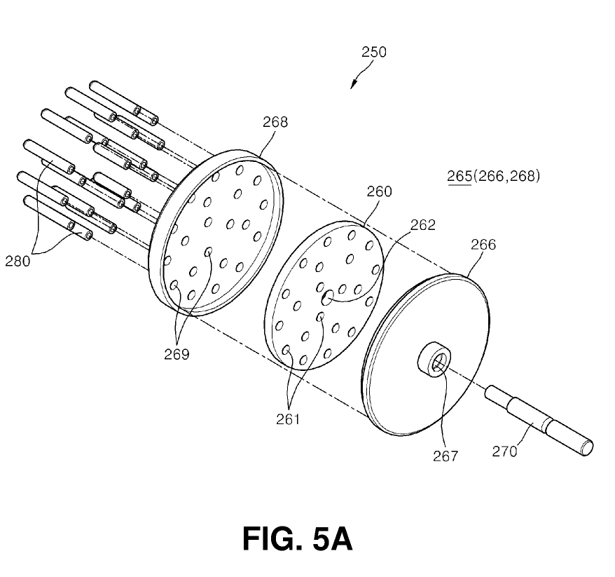

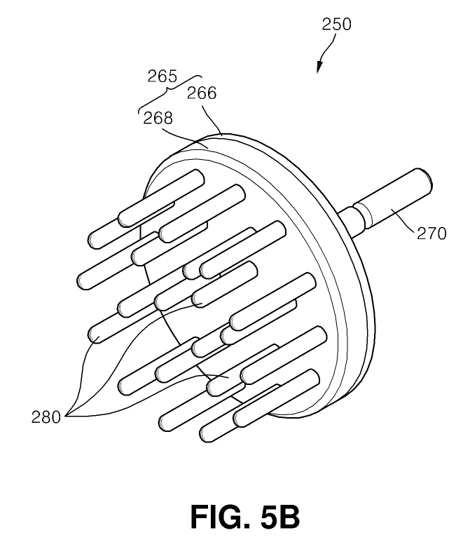

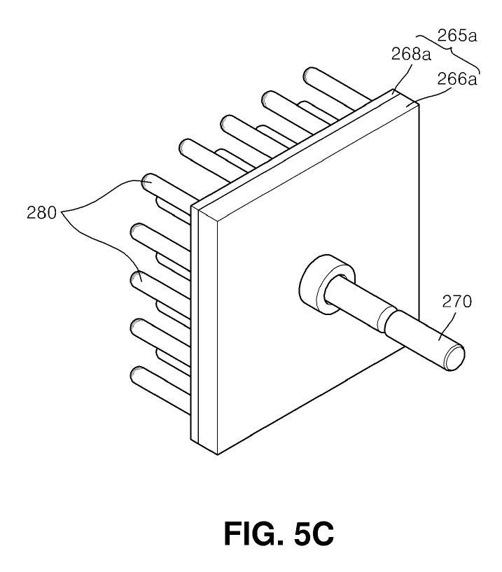

FIG. 5a is an exploded perspective view illustrating a head portion of the present invention, FIG. 5b is an assembled perspective view illustrating a head portion of the present invention, and FIG. 5c is an assembled perspective view illustrating a modification example of a head portion of the present invention.

Referring to FIGS. 5a and 5b, the head portion 250 includes a base electrode plate 260, a first insulator 265, a connecting terminal 270 and a plurality of protruding terminals 280. The first insulator 265 includes an upper case 266 and a lower case 268.

The base electrode plate 260 is formed in a disc shape in its entirety, and has a plurality of peripheral through-holes 261 and a central through-hole 262, which are formed therein. The peripheral through-holes 261 are arranged spaced apart from one another, and are formed spaced apart at predetermined intervals from the center thereof. That is, the peripheral through-holes 261 are formed in a radial direction on the base electrode plate 260. The base electrode plate 260 is made of a conductive material for conduction of the high-frequency current. In this embodiment, the base electrode plate 260 is made of copper, but is not limited thereto.

The first insulator 265 includes an upper case 266 disposed on an upper portion of the base electrode plate, and a lower case 268 disposed on a lower portion of the base electrode plate and coupled to the upper case 266. The lower case 268 has a plurality of peripheral through-holes 269 formed therein so as to correspond to the plurality of peripheral through-holes 261 formed in the base electrode plate 260, and the upper cases 266 has a through-hole 267 formed at the center thereof. The connecting terminal 270 passes through the through-hole 267 of the upper case 266 so as to be fittingly engaged with the central through-hole 262 of the base electrode plate 260.

The plurality of protruding terminals 280 is fittingly engaged with the peripheral through-holes 261 formed in the base electrode plate 260, and is formed to be divided into a plurality of groups of different lengths. Among the plurality of protruding terminals 280, longer protruding terminals are arranged nearer to the outer peripheral edge of the base electrode plate 260 and shorter protruding terminals are arranged nearer to the central region of the base electrode plate 260, so that the entire bottom contour of the plurality of the thus arranged protruding terminals 280 has an upwardly concave shape at the center thereof. In this manner, if the plurality of protruding terminals 280 is constructed such that the lengths thereof are made different, all the protruding terminals 280 evenly come into direct close contact with the affected part of the user, which results in an increase in the contact area with the scalp to thereby more efficiently perform the stimulation of the scalp.

The head portion shown in FIG. 5c is different from the head portion as described in the above embodiment in that it is formed in a square shape in its entirety, and is similar to the head portion of the above embodiment in terms of the remaining constructions. Thus, the detailed description of the head portion of FIG. 5c will be made focusing on the difference in construction. The head portion shown in FIG. 5c is formed in a square plate shape in its entirety. A base electrode plate (not shown) and a first insulator 265a adapted to surround the base electrode plate are also formed in a square plate shape.

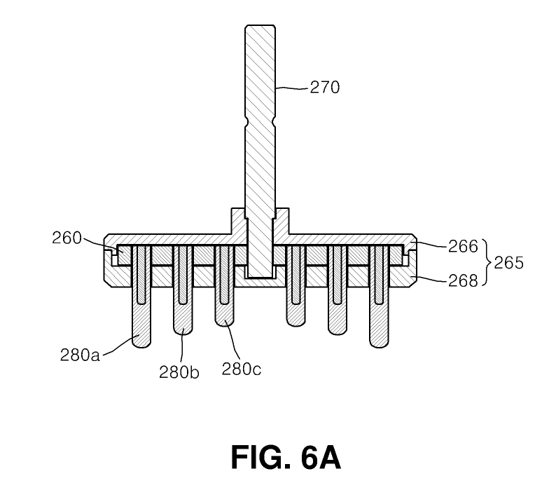

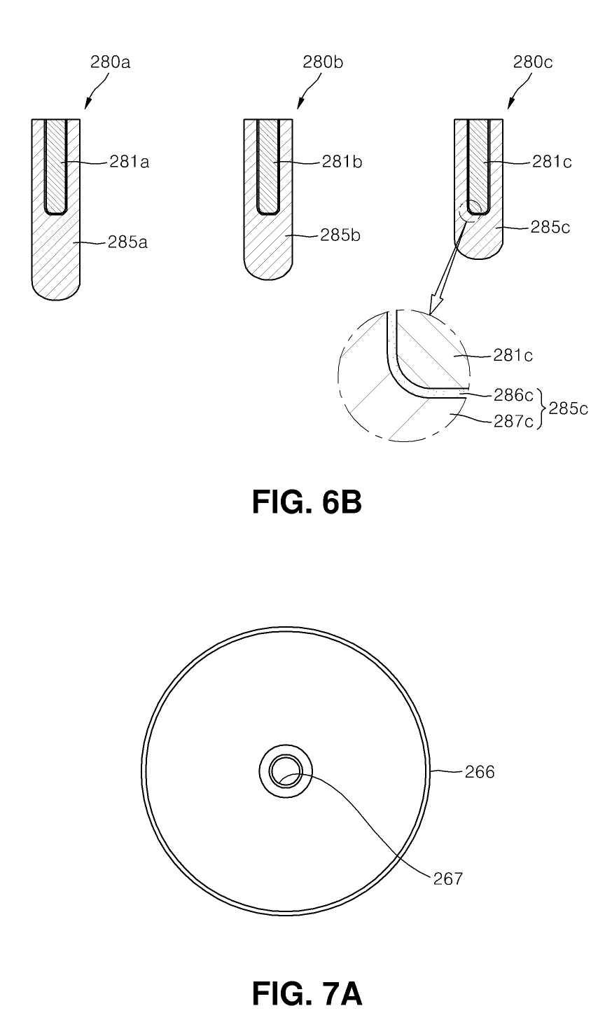

FIG. 6a is a schematic vertical cross-sectional view illustrating a head portion of the present invention, and FIG. 6b is a schematic vertical cross-sectional view illustrating a plurality of protruding terminals of the head portion of the present invention.

Referring to FIGS. 6a and 6b, the plurality of protruding terminals 280 consist of a first group of protruding terminals 280a, a second group of protruding terminals 280b and a third group of protruding terminals 280c. The first group of protruding terminals 280a are arranged nearer to the outer peripheral edge of the base electrode plate 260, the third group of protruding terminals 280c are arranged nearer to the central region of the base electrode plate 260, and the second group of protruding terminals 280b are arranged between the first group of protruding terminals 280a and the third group of protruding terminals 280c. The length of the protruding terminal is gradually increased in the order of the first group of protruding terminals 280a, the second group of protruding terminals 280b and the third group of protruding terminals 280c, so that the entire bottom contour of the plurality of the thus arranged protruding terminals 280 has an upwardly concave shape at the center thereof.

Each of the protruding terminals 280 includes a conductor 281 and a second insulator 285 adapted to surround the conductor 281. In this embodiment, the conductor 281 is made of aluminum, but is not limited thereto and may be made of various materials. As shown in FIG. 6b, in order to make the lengths of the plurality of protruding terminals different, the first to third groups of protruding terminals 280a, 280b and 280c can be constructed such that the lengths of the respective conductors 281a, 281b and 281c are made the same, and the coating thicknesses of the second insulators 285a, 285b and 285c are made different. Alternatively, in order to make the lengths of the plurality of protruding terminals different, the first to third groups of protruding terminals 280a, 280b and 280c may be constructed such that the lengths of the respective conductors are made different, and the coating thicknesses of the second insulators are made the same.

Now, even if the construction of the second insulator 285 will be described by example of the third group of protruding terminals 280c, the second insulators of the remaining first and second groups of protruding terminals 280a and 280b are also constructed in the same manner as in the third group of protruding terminals 280c.

The second insulator 285c includes a first layer 286c surroundingly coated on the conductor 281c, and a second layer 287c coated on the first layer 286c. The first layer 286c is formed on the conductor 281c by means of a ceramic coating method, and the second layer 287c is formed on the first layer 286c by means of a nylon coating method. In this manner, when the conductor 281c is doubly coated, a crack can be prevented from occurring, and as a consequence, the high-frequency current can be prevented from being directly applied to the human body, thereby further improving stability and reliability of the product.

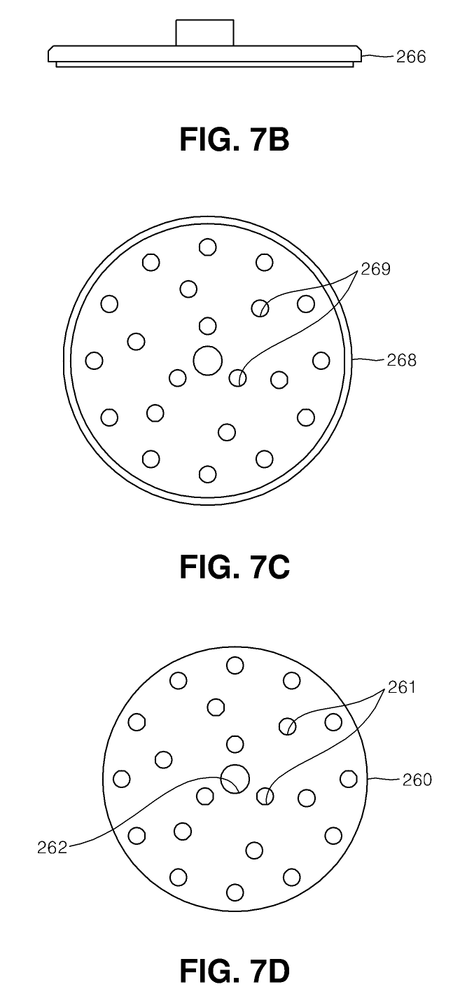

FIG. 7a is a top plan view illustrating an upper case of a first insulator of the head portion of the present invention, FIG. 7b is a side view illustrating the upper case of the first insulator of the present invention, FIG. 7c is a top plan view illustrating a lower upper case of the first insulator of the present invention, and FIG. 7d is a top plan view illustrating a base electrode plate of the head portion of the present invention.

Referring to FIGS. 7a to 7d, the upper case 266 of the first insulator is formed in a disc shape which is protruded at the central portion thereof, and the through-hole 267 is centrally formed in the protruded portion so as to provide a space for allowing the connecting terminal 270 to be inserted thereto.

The base electrode plate 260 has a central through-hole 262 formed at the central region thereof, and a plurality of peripheral through-holes 261 formed around the central through-hole 262, i.e., at the peripheral region thereof in such a fashion as to be spaced apart from one another at predetermined intervals. Three through-holes are formed adjacent to the central through-hole 262, six through-holes are circumferentially formed around the three through-holes, and twelve through-holes are circumferentially formed adjacent to the outer peripheral edge of the base electrode plate 260, so that the plurality of through-holes is formed in a radial shape in its entirety.

The lower case 268 has a plurality of through-holes 269 formed at the peripheral region thereof so as to correspond to the sizes and positions of the plurality of through-holes 261 formed at the base electrode plate 260 so that a space for inserting a plurality of protruding terminals (not shown) thereto is provided. The lower case 268 has an insertion recess 269b formed at the central region thereof so as to allow one end of the connecting terminal 270 to be inserted thereto.

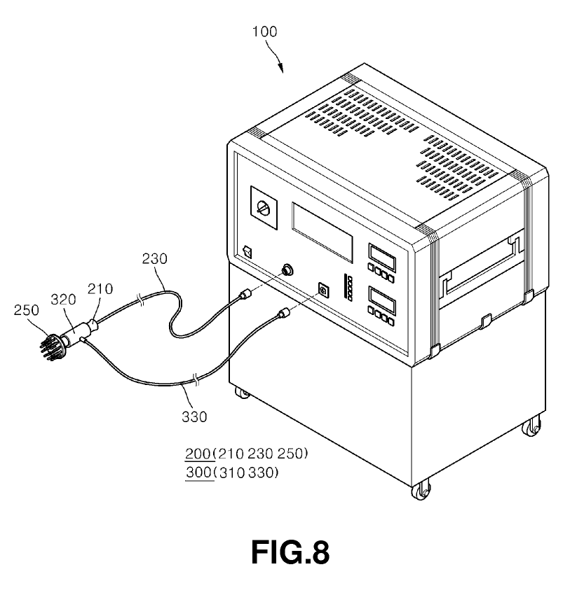

FIG. 8 is a top perspective view illustrating a high-frequency treatment apparatus for scalp care according to another preferred embodiment of the present invention.

An embodiment shown in FIG. 8 is different from the above embodiment in that the second electrode unit is mounted on a part of the first electrode unit. Thus, the detailed description of the embodiment of FIG. 8 will be made focusing on the difference in construction.

Referring to FIG. 8, a high-frequency treatment apparatus for hair care according to another preferred embodiment of the present invention includes: a high-frequency generation unit 100 for generating a high-frequency current: a first electrode unit 200 electrically connected to the high-frequency generation unit, the first electrode unit including a plurality of protruding terminals adapted to come into close contact with a scalp of a user to be treated and being configured to apply the high-frequency current generated from the high-frequency generation unit to the scalp through the plurality of protruding terminals; and a second electrode unit 300 electrically connected to the high-frequency generation unit, the second electrode unit including an electrode plate adapted to come into close contact with another body part of the user and being configured to allow the high-frequency current applied to the scalp to be conducted to the contacted other body part of the user through the electrode plate.

The first electrode unit 200 includes a body portion 210, a first cable 230 and a head portion 250, and the second electrode unit 300 includes a second electrode plate 320 and a second cable 330. The second electrode plate 320 is mounted on the body portion 210 of the first electrode unit 200. As a result, when the user or user contacts the head portion 250 with an affected part to be treated, i.e., a scalp by gripping the body portion 210 of the first electrode unit 200 with his or her hand, the user's hand gripping the body portion 210 also comes into close contact with the second electrode plate 320, so that the high-frequency treatment can be performed smoothly even without doing a secondary behavior in which a separate second electrode plate 320 should come into close contact with another part of the human body.

The electrode unit for scalp care according to the present invention has the following advantageous effects.

First, the electrode unit for scalp care according to the present invention supplies a high-frequency current to a scalp to be treated through a plurality of protruding terminals so that the protruding terminals and the scalp can be easily come into direct close contact with each other, thereby reducing the treatment time.

Second, the high-frequency treatment apparatus for scalp care according to the present invention is provided with an electrode unit including a plurality of protruding terminals having different lengths to conform to the contour of the human head, thereby increasing the contact area with the scalp.

Third, the electrode unit for scalp care according to the present invention employs a plurality of protruding terminals so as to allow the deep heat to be produced around the scalp to which the high-frequency current is applied and simultaneously stimulate the scalp through the protruding terminals, thereby maximizing a hair and scalp care effect.

While the present invention have been described in connection with the exemplary embodiments illustrated in the drawings, they are merely illustrative embodiments of the electrode unit for scalp care, and the invention is not limited to these embodiments. It is to be understood that various equivalent modifications and variations of the embodiments can be made by a person having an ordinary skill in the art without departing from the spirit and scope of the present invention. Therefore, the true technical scope of the present invention should be defined by the appended claims.

What is claimed is:

1. An electrode unit for scalp care which is adapted to comes into close contact with a scalp of a user to be treated and apply a high-frequency current to the scalp so as to produce deep heat and treat the scalp by using the produced deep heat, the electrode unit comprising:
a flat-shaped base electrode plate;
a connecting terminal which is formed at one side of the base electrode plate and to which the high-frequency current is inputted; and
a plurality of protruding terminals which is formed at the other side of the base electrode plate and is adapted to allow the high-frequency current inputted to the connecting terminal to be applied to the scalp therethrough.

2. The high-frequency electrode unit for scalp care according to claim 1, which further comprises a first insulator adapted to surround the base electrode plate, and wherein each of the plurality of protruding terminals comprises a conductor and a second insulator adapted to surround the conductor.

3. The high-frequency electrode unit for scalp care according to claim 1, wherein the base electrode plate comprises a first engagement through-hole formed at the central region thereof and a plurality of second engagement through-holes formed at the peripheral regions thereof, wherein the connecting terminal further comprises a first engagement portion fittingly engaged with the first engagement through-hole of the base electrode plate and a second, and wherein each of the plurality of protruding terminals further comprises a second engagement portion fittingly engaged with the second engagement through-holes of the base electrode plate.

4. The high-frequency electrode unit for scalp care according to claim 1, wherein the base electrode plate, and connecting terminal and the plurality of protruding terminals are formed integrally with one another.

5. The high-frequency electrode unit for scalp care according to claim 2, wherein the first insulator comprises:
an upper case adapted to protectively cover an upper portion of the base electrode plate; and
a lower case coupled to the upper case and adapted to protectively cover a lower portion of the base electrode plate.

6. The high-frequency electrode unit for scalp care according to claim 1, wherein the plurality of protruding terminals is arranged in such a fashion as to be spaced apart from one another, and the lengths of the plurality of protruding terminals are gradually reduced as it goes toward the center of the base electrode plate.

7. The high-frequency electrode unit for scalp care according to claim 2, wherein the second insulator comprises:
a first layer surroundingly coated on the conductor; and
a second layer coated on the first layer, and
wherein the first layer comprises a ceramic material and the second layer comprises a nylon material.

8. A high-frequency treatment apparatus for scalp care, comprising:
a high-frequency generation unit for generating a high-frequency current:
a first electrode unit electrically connected to the high-frequency generation unit, the first electrode unit including a plurality of protruding terminals adapted to come into close contact with a scalp of a user to be treated and being configured to apply the high-frequency current generated from the high-frequency generation unit to the scalp through the plurality of protruding terminals; and
a second electrode unit electrically connected to the high-frequency generation unit, the second electrode unit including an electrode plate adapted to come into close contact with another body part of the user and being configured to allow the high-frequency current applied to the scalp to conducted to the contacted other body part of the user through the electrode plate.

9. The a high-frequency treatment apparatus for scalp care according to claim 8, wherein the second electrode unit is mounted on the body portion of the first electrode unit.

10. The a high-frequency treatment apparatus for scalp care according to claim 8, wherein the high-frequency current has a frequency of 400 kHz to 600 kHz.
